(19) United States
(12) Patent Application Publication
(10) Pub. No.: US 2006/0271081 A1
(43) Pub. Date: Nov. 30, 2006

(54) Apparatus and methods for valve repair

(76) Inventor: Fidel Realyvasquez, Palo Cedro, CA (US)

Correspondence Address: HELLER EHRMAN LLP, 275 MIDDLEFIELD ROAD, MENLO PARK, CA, 94025-3506, US

(21) Appl. No.: 10/551,856

(22) PCT Filed: Mar. 30, 2004
(86) PCT No.: PCT/US04/09790

§ 371 Date: Jul. 11, 2006

Related U.S. Application Data
(60) Provisional application No. 60/459,385, filed on Mar. 30, 2003.
(60) Provisional application No. 60/459,560, filed on Mar. 30, 2003.

(51) Int. Cl. A61B 17/3205; A61F 2/24

(52) U.S. Cl. 606/170; 623/2.11; 623/2.38

(57) ABSTRACT

According to one aspect, heart valve leaflet removal apparatus (100) of the present invention comprises a pair of cooperating cutting elements (112), a holder and members for manipulating the cutting elements (112). The cooperating cutting elements (112) are adapted for cutting and removing leaflets from an aortic valve in a patient's heart, one of the cutting elements (112) is rotatably coupled the other of the pair of cutting elements (112). The holder is coupled to one of the cutting elements and is adapted to receive the cut leaflets and the cutting elements and holder are configured for delivery to the valve leaflets through an aortotomy formed in a patient's aorta. In one variation, the pair of cooperating cutting elements (112) and holder have a radial dimension and are radially collapsible. Replacement valve delivery apparatus also is provided.

### FIELD OF THE INVENTION

The invention relates to apparatus and methods for valve replacement and is especially useful in aortic valve repair procedures.

### BACKGROUND OF THE INVENTION

Essential to normal heart function are four heart valves, which allow blood to pass through the four chambers of the heart in one direction. The valves have either two or three cusps, flaps, or leaflets, which comprise fibrous tissue that attaches to the walls of the heart. The cusps open when the blood flow is flowing correctly and then close to form a tight seal to prevent backflow.

The four chambers are known as the right and left atria (upper chambers) and right and left ventricles (lower chambers). The four valves that control blood flow are known as the tricuspid, mitral, pulmonary, and aortic valves. In a normally functioning heart, the tricuspid valve allows one-way flow of deoxygenated blood from the right upper chamber (right atrium) to the right lower chamber (right ventricle). When the right ventricle contracts, the pulmonary valve allows one-way blood flow from the right ventricle to the pulmonary artery, which carries the deoxygenated blood to the lungs. The mitral valve, also a one-way valve, allows oxygenated blood, which has returned to the left upper chamber (left atrium), to flow to the left lower chamber (left ventricle). When the left ventricle contracts, the oxygenated blood is pumped through the aortic valve to the aorta.

Certain heart abnormalities result from heart valve defects, such as valvular insufficiency. Valvular insufficiency is a common cardiac abnormality where the valve leaflets do not completely close. This allows regurgitation (i.e., backward leakage of blood at a heart valve). Such regurgitation requires the heart to work harder as it must pump both the regular volume of blood and the blood that has regurgitated. If this insufficiency is not corrected, the added workload can eventually result in heart failure.

Another valve defect or disease, which typically occurs in the aortic valve is stenosis or calcification. This involves calcium buildup in the valve which impedes proper valve leaflet movement.

In the case of aortic valve insufficiency or stenosis, treatment typically involves removal of the leaflets and replacement with valve prosthesis. However, known procedures have involved generally complicated approaches that can result in the patient being on cardiopulmonary bypass for an extended period of time.

Applicants believe that there remains a need for improved valvular repair apparatus and methods that use minimally invasive techniques and/or reduce time in surgery.

Mitral valve insufficiency can also be problematic. Various approaches to correct mitral valve defects have included valve replacement, valve leaflet repair, chordae tendineae shortening or replacement, and or valve annulus repair also known as annuloplasty. One example where annuloplasty procedures have been developed is in the field of mitral valve insufficiency correction.

Mitral valve insufficiency typically results from a change in the size and shape of the mitral valve annulus. Mitral valve annuloplasty involves reestablishing the normal shape and size of the mitral valve annulus so that it can effect full closure of the valve leaflets.

Approaches to improve valve function (e.g., mitral or tricuspid valve function) have included tissue plication devices and reinforcement of the valve annulus with annuloplasty rings. These approaches have been stated to reestablish the original annulus size and shape and/or prevent further annulus dilation.

Both rigid and flexible annuloplasty rings have been developed. Rigid rings, which generally tend to dictate the shape and contour of the mitral valve annulus, have been considered to somewhat compromise the natural flexibility of the annulus. Flexible annuloplasty rings emerged to provide some degree of compliance in the valve annulus so that the valve could maintain normal physiological motion throughout the cardiac cycle of a beating heart. This is in addition to providing annulus reinforcement. However, it is believed that among the drawbacks of these rings is that they may fold or crimp during implantation and thereby undesirably reduce the size of the valve (e.g., the mitral valve) opening. Also, the sutures used to secure the ring may cause scarring and stiffening of the valve annulus and reduce annulus flexibility over time.

C-shaped bands or partial annuloplasty rings also have been developed. These devices can be attached solely to the posterior portion of the valve annulus which eliminates the need to attach material to the anterior portion of the annulus. Full and partial ring devices are disclosed, for example, in U.S. Pat. No. 3,656,185, which issued to Carpentier.

Other attempts to improve upon valve repair procedures include those described in U.S. Pat. No. 5,450,860, which issued to O'Connor, U.S. Pat. No. 6,183,512, which issued to Howanec, Jr. et al., and U.S. Pat. No. 6,250,308, which issued to Cox.

Applicants believe that there remains a need for improved valvular repair apparatus and methods

### SUMMARY OF THE INVENTION

The present invention involves valve repair apparatus and methods that overcome problems and disadvantages of the prior art. According to one aspect of the invention, minimally invasive valve removal apparatus is provided, which includes cutting elements configured for delivery to the valve through an aortotomy formed in the patient's aorta. Other aspects of the invention include, but are not limited to replacement valve delivery apparatus.

In one embodiment of the invention, heart valve leaflet removal apparatus comprises a pair of cooperating cutting elements, a holder and members for manipulating the cutting elements. The cooperating cutting elements are adapted for cutting and removing leaflets from an aortic valve in a patient's heart and one of the cutting elements is rotatably coupled the other of the pair of cutting elements. The holder is coupled to one of the cutting elements and is adapted to receive the cut leaflets and the cutting elements and holder are configured for delivery to the aortic valve leaflets through an aortotomy formed in the patient's aorta. In one variation, the pair of cooperating cutting elements and holder have a radial dimension and are radially collapsible.

According to another embodiment of the invention, a heart valve repair system comprises heart valve leaflet removal apparatus comprising a pair of cooperating cutting elements adapted for cutting and removing leaflets from an aortic valve in a patient's heart, one of the cutting elements being rotatably coupled to the other of the pair of cutting elements, a holder coupled to one of the cutting elements and adapted to receive the cut leaflets, the cutting elements and holder being configured for delivery to the aortic valve leaflets through an aortotomy formed in the patient's aorta; and heart valve prosthesis delivery apparatus for placing an aortic valve prosthesis in the patient's heart comprising an aortic valve prosthesis support having a proximal portion and a distal portion and a plurality of fasteners ejectably mounted therein, the distal portion being adapted to be releasably coupled to the aortic valve prosthesis, and the valve prosthesis support being configured for delivery to the heart through the aortotomy formed in the patient's aorta.

According to another embodiment of the invention, a replacement valve delivery system comprises heart valve prosthesis delivery apparatus for placing an aortic stentless valve prosthesis in a patient's heart comprising an aortic stentless valve prosthesis support having a proximal portion and a distal portion and a plurality of fasteners ejectably mounted therein, the distal portion being adapted to be releasably coupled the aortic valve prosthesis, and the valve prosthesis support being configured for delivery to the heart through an aortotomy formed in the patient's aorta; and a balloon adapted to be placed in the valve prosthesis and urge at least a portion of the valve prosthesis against the inner wall of the aorta of the patient so that when adhesive is applied to an exterior portion of the valve prosthesis and the valve prosthesis urged against the inner wall of the aorta the exterior portion can adhere to the inner wall of the aorta

According to another embodiment of the invention, a method of repairing an aortic valve comprises removing aortic leaflets form a patient's aortic valve; providing valve prosthesis on delivery apparatus where the valve prosthesis has an annular portion; introducing the valve prosthesis through an aortotomy formed in the patient's aorta with the delivery apparatus; simultaneously ejecting a plurality of self-closing clips from the delivery apparatus through said annular portion and then into the patient's aortic root to secure the valve prosthesis to the aortic root of the patient.

In yet another embodiment of the invention, heart valve prosthesis includes a curved member and a skirt. The curved member can have first and second ends and be adapted to form a partial ring along a portion of one of the valve annulae in the patient's heart. Alternatively, the curved member can form a fall ring that is adapted to extend along the entire valve annulus. The skirt extends along the curved member and depends therefrom. This prosthesis is especially useful in treating mitral valve insufficiency. In this case, the skirt can be configured so that when the prosthesis is secured to the mitral valve along the mitral valve annulus, the skirt covers the posterior leaflet and the opposed edges of the skirt and anterior leaflet coapt. In addition, when the curved member is secured to the posterior portion of the mitral valve annulus, further annulus dilation can be minimized or eliminated.

According to another embodiment of the invention, heart valve delivery apparatus for placing heart valve prosthesis in a patient's heart comprises a delivery device comprising a plurality of tube pairs arranged to support the heart valve prosthesis; and a plurality of self-closing clips, each clip having an open configuration and a closed configuration and first and second piercing ends, each clip being ejectably mounted to one of the tube pairs with a first portion of the clip slidably postioned in one tube of the tube pair and a second portion slidably postioned in the other tube of the tube pair so that the first clip piercing end can be ejected from the one tube of the tube pair and the second piercing end can be ejected from the other tube of the tube pair.

According to another embodiment of the invention, heart valve repair apparatus for placing heart valve prosthesis in a patient's heart comprises heart valve prosthesis comprising a prosthetic valve leaflet and a member supporting the leaflet; delivery apparatus comprising a support for the valve prosthesis and a plurality of clips ejectably mounted to the delivery apparatus support, each clip having two piercing tips extending into the member supporting the leaflet.

According to another embodiment of the invention, a method for delivering heart valve prosthesis comprises providing heart valve prosthesis having a curved member and a skirt extending therefrom and a plurality of self-closing clips, each having two pointed ends, and an open configuration and a closed configuration; securing the curved member to said plurality of self-closing clips with the two pointed ends of each clip penetrated into the curved member; placing the curved member on the mitral valve annulus of a patient's heart; ejecting all of the clips simultaneously to penetrate into the valve annulus and move toward their closed configuration to secure the heart valve prosthesis to the valve annulus. The above is a brief description of some deficiencies in the prior art and advantages of the present invention. Other features, advantages, and embodiments of the invention will be apparent to those skilled in the art from the following description and accompanying drawings, wherein, for purposes of illustration only, specific forms of the invention are set forth in detail.

### BRIEF DESCRIPTION OF THE DRAWINGS

FIG. 1 illustrates an aortic root pulled back to show the aortic valve leaflets to be removed in an aortic valve replacement procedure of the present invention;

FIG. 2A is perspective view of minimally invasive valve cutting apparatus suitable for removing the valve leaflets from an aortic valve in accordance with the present invention and shown in a collapsed state;

FIG. 2B is a perspective view of the apparatus of FIG. 2A shown in an expanded state and illustrated for exemplary purposes positioned in an aortic valve;

FIG. 2C is a perspective view of the apparatus of FIG. 2B illustrating the cutting members of the apparatus engaged after cutting the aortic valve leaflets from the aortic valve;

FIG. 3A is a perspective view of another minimally invasive valve cutting apparatus in accordance with the present invention;

FIGS. 3B, 3C, and 3D are diagrammatic partial sectional views of the apparatus of FIG. 3A where FIG. 3B shows the pair of cooperating cutting elements of the apparatus above the valve leaflets, FIG. 3C shows one of the cooperating cutting elements positioned below the valve leaflets, and FIG. 3D shows the upper cooperating cutting element rotated and the valve leaflets separated form the original valve;

FIG. 4A is a perspective view of valve prosthesis and clip delivery apparatus in accordance with the invention shown supporting valve prosthesis and being in a collapsed state for minimally invasive delivery of the valve prosthesis (e.g., through an aortotomy);

FIG. 4B is another perspective view of the delivery apparatus of FIG. 4A with the support arm slide retracted to place the arms in an expanded state;

FIG. 4C is another perspective view of the delivery apparatus of FIG. 4A with the clip ejection actuator moved distally to eject the fasteners, which fasten the valve prosthesis to the surgical site;

FIG. 4D is another perspective view of the delivery apparatus of FIG. 4A illustrating removal of the delivery apparatus after the clips have been released;

FIGS. 5A-5D are partial sectional views of the distal end of the delivery apparatus of FIG. 4A and the valve prosthesis seated on an aortic valve diagrammatically illustrating clip delivery where FIG. 5A shows the ends of the support arms penetrated through the sides of the replacement valve, FIG. 5B shows the ejection of the clips into the aortic root wall, FIG. 5C illustrates withdrawal of the ends of the support arms and the clips fully released and securing the valve prosthesis to the aortic valve annulus, and FIG. 5D illustrates complete removal of the prosthesis and clip delivery apparatus;

FIG. 5E is a detailed view illustrating a pusher member of the valve prosthesis and clip delivery apparatus ejecting a clip;

FIG. 5F illustrates the clip of FIG. 5E discharges from the delivery apparatus support arm and in place where it secures a portion of the valve prosthesis to the aortic annulus;

FIG. 6 illustrates how the valve prosthesis attachment would appear if the aortic root were cut and pulled back after implantation;

FIG. 7 illustrates placement of an expandable balloon within the valve prosthesis after the valve prosthesis is secured to the aortic annulus with the balloon expanded and compressing the outer wall surfaces of prosthesis having bio-glue applied thereto against the aortic inner wall;

FIG. 8 is a perspective view of the delivery apparatus of FIG. 4A supporting a mechanical valve;

FIG. 9A is a side view of the mechanical valve of FIG. 8 in an open state;

FIG. 9B is a side view of the mechanical valve of FIG. 8 in a closed state;

FIG. 10 is a perspective view of the mechanical valve secured to the aortic annulus after delivery with the delivery apparatus of FIG. 9; and

FIG. 11 is a top plan view the fastener clip depicted in various of the foregoing FIGS. shown in a relaxed or free state.

FIG. 12A is a perspective view of one embodiment of a valve prosthesis in accordance with the principles of the present invention with the prosthetic leaflet in a closed position;

FIG. 12B is a perspective view of the valve prosthesis of FIG. 12A with the prosthetic leaflet in an open position;

FIG. 13A is a top plan view of the valve prosthesis of FIG. 12A;

FIG. 13B is a top plan view of the valve prosthesis of FIG. 12B;

FIG. 14A is a side elevational view of the prosthesis of FIG. 12A;

FIG. 14B is a side elevational view of the prosthesis of FIG. 12B;

FIGS. 15A-C are partial sectional views of a clip delivery mechanism for securing the prosthesis of FIG. 12A to a patient's valve where FIG. 15A depicts the clip in a first loaded position, FIG. 15B depicts the clip in an intermediate position, and FIG. 15C depicts the clip ejected from the delivery mechanism;

FIGS. 16A-C are longitudinal partial cross sections of the clip delivery mechanism of FIGS. 15A-C where FIG. 16A depicts the clip in a first loaded position, FIG. 16B depicts the clip in an intermediate position, and FIG. 16C depicts the clip ejected from the delivery mechanism;

FIGS. 17A-C are partial cross sections of the clip delivery mechanism of FIGS. 16A-C rotated 90 degrees where FIG. 17A is taken along line 17A-17A of FIG. 16A illustrating the clip in a first loaded position, FIG. 17B depicts the clip of FIG. 17A in an intermediate position, and FIG. 17C depicts the clip of FIG. 17A ejected from the delivery mechanism;

FIG. 18 is a perspective view of prosthesis delivery apparatus, which in the illustrative embodiment, includes a plurality of the delivery mechanisms of FIG. 15A-C;

FIGS. 19A-19E illustrate delivery and securement of the prosthesis of FIG. 12 using the prosthesis delivery mechanism of FIG. 18 where FIG. 19A is a perspective view of the prosthesis delivery apparatus of FIG. 18 and the prosthesis of FIG. 12A secured thereto and positioned for securement to a mitral valve annulus, FIG. 19B illustrates the prosthesis delivery mechanism of FIG. 18 seated on the valve annulus; FIG. 19C illustrates simultaneous ejection of all of the clips from the clip delivery mechanisms with a single actuation mechanism, FIG. 19D illustrates the clips securing the valve prosthesis in place along the valve annulus and removal of the prosthesis delivery apparatus, and FIG. 19E illustrates a top view of the valve prosthesis in place over the mitral valve with the anterior leaflet in view and in a closed position and with the prosthetic leaflet or skirt covering the posterior leaflet.

### DETAILED DESCRIPTION OF THE INVENTION

Before the present invention is described, it is to be understood that this invention is not limited to the particular embodiments or examples described, as such may, of course, vary. Further, when referring to the drawings, like numerals indicate like elements.

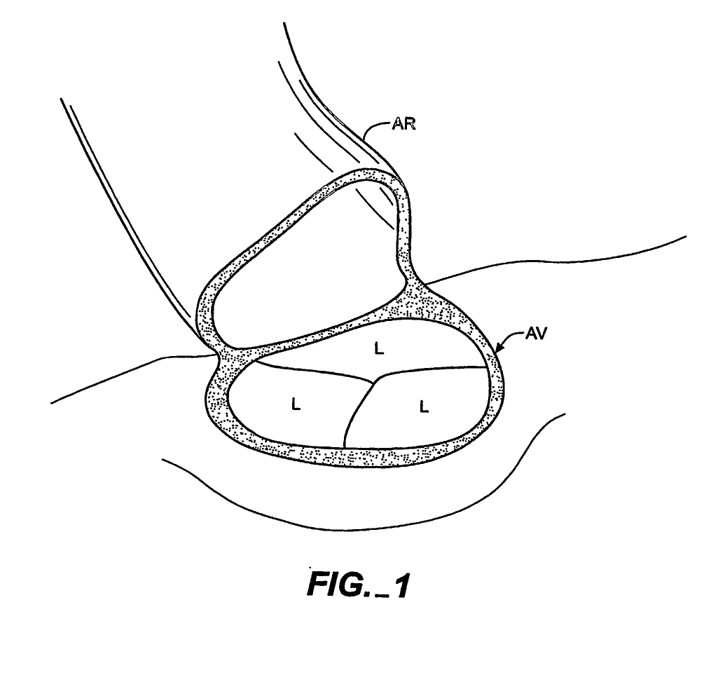

Referring to FIG. 1, an aortic root (AR) is shown pulled back to show the right, left, and posterior leaflets (L) of an aortic valve (AV) to be removed in a minimally invasive valve replacement procedure of the present invention where valve leaflet removal apparatus and valve prosthesis delivery apparatus can be delivered to the aortic root via an aortotomy.

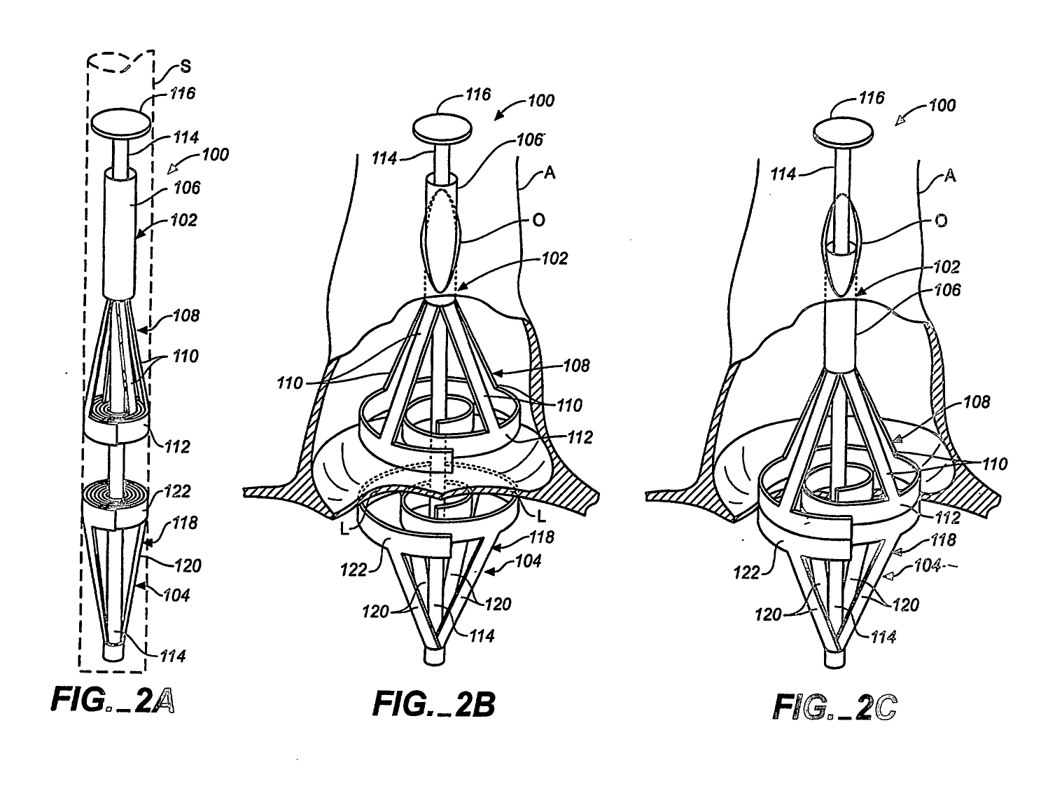

Referring to FIGS. 2A-C, one embodiment of minimally invasive valve cutting or removal apparatus is shown and generally designated with reference numeral 100. Apparatus 100 includes a first body member 102 and a second body member 104. First body member 102 includes a tubular member 106 and an umbrella 108 having umbrella arms 110 and a cutting element 112, which is in the form of a spiral. Cutting element 112 can be formed from flat metal wire, such as flat stainless steel wire or ribbon or any other materials suitable for cutting. Umbrella arms 110 each have one end secured to or integrally formed with tubular member 106 and one end secured to or integrally formed with cutting element 112.

Second body member 104 includes an elongated member 114, which can include a knob 116 at one end thereof. Second body member 104 also includes an umbrella 118, which is similar to umbrella 108. Umbrella 118 includes umbrella arms 120 and umbrella cutting element 122, which also is in the form of a spiral. Cutting element 122 can be formed from flat metal wire, such as flat stainless steel wire or ribbon or any other material suitable for cutting. Umbrella arms 120 each have one end secured to or integrally formed with elongated member 114 and one end secured to or integrally formed with cutting element 122.

As shown in FIG. 2A, the first and second umbrellas 108 and 118 are radially compressible or collapsible. A tube or sheath such as shown in dashed lines and indicated with reference character “S” in FIG. 2A can be placed around apparatus 100 to hold it in a collapsed state (i.e., with umbrellas 108 and 118 radially compressed). With the sheath in place so that the umbrellas are in the radially compressed or collapsed state, where the umbrellas have a radial dimension less that of their uncompressed or uncollapsed state as shown in FIG. 2B, sheath S and valve removal apparatus 100 are introduced through an opening 0 or aortotomy formed in the aorta (A) of a patient. When the second umbrella is positioned below the aortic leaflets (L) and the first umbrella is positioned above the aortic leaflets (L), the umbrellas are allowed to expand to their memory or relaxed state shown in FIG. 2B by retracting the sheath. The expanded memory shape of FIG. 2B can be provided by heat treating stainless steel flat wire or other suitable material in the desired expanded configuration as is known in the art. If the umbrellas are not aligned as shown in FIG. 2A, members 106 and 114 can be manipulated to adjust the umbrella positions. Other mechanisms for holding elements 112 and 122 or for holding the umbrellas radially compressed can be used. For example, a wire can be wrapped around elements 112 and 122 and pulled away from the apparatus when the umbrellas are in place and ready to deploy.

Referring to FIG. 2C, tubular member 106 and elongated member 114 are then moved in opposite directions toward one another to compress the leaflets between the opposed cutting edges of cutting elements 112 and 122, which edges can be sharpened to enhance cutting. Tubular member 106 and/or elongated member 114 also can be rotated to complete the cut if necessary. The cut leaflets can fall into second umbrella 118, which forms a holder for the leaflets if they do not remain between the cutting edges during removal of the apparatus.

Before removing apparatus 100, it again is radially compressed. This can be done by sliding sheath S over apparatus 100. If the second umbrella does not close with the first umbrella, i.e., if the sheath does not readily slide over the second umbrella, the surgeon can retract the apparatus so that the second umbrella is in the vicinity of the aortotomy and manipulate spiral cutting element 122 to reduce the diameter of the second umbrella. The manual manipulation of element 122 can facilitate sliding the sheath thereover or facilitate pulling the unsheathed second umbrella through the aortotomy. In this manner, apparatus 100, together with the cut leaflets are removed from the site through the aortotomy.

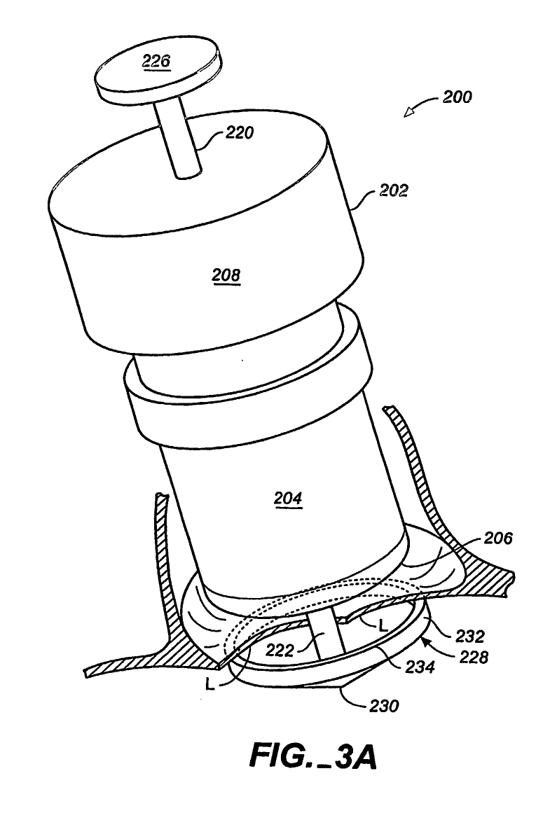

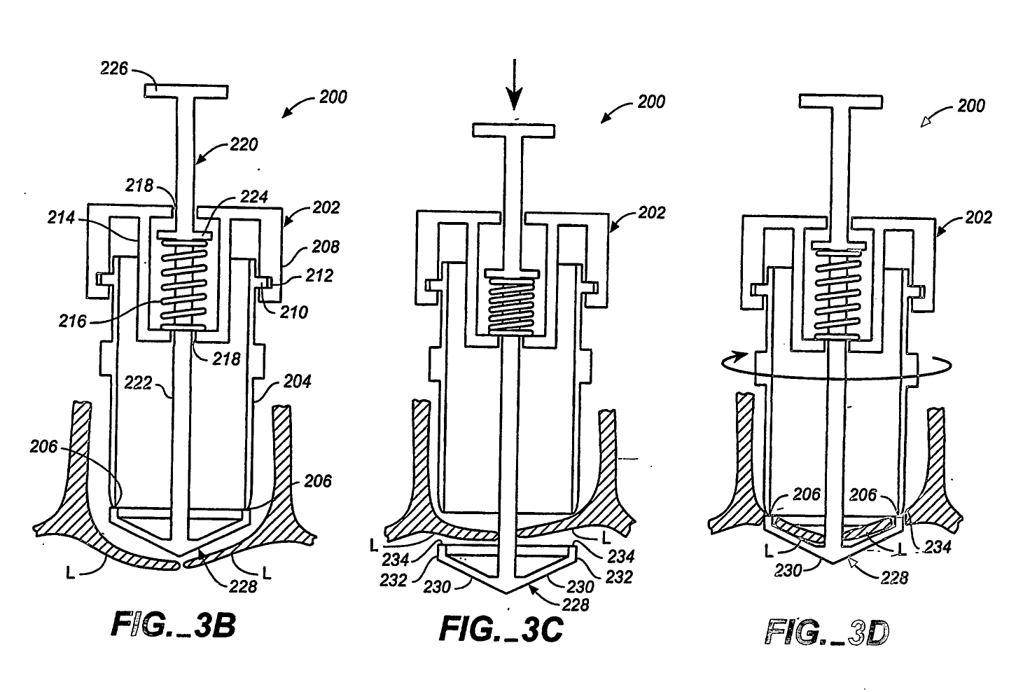

Referring to FIGS. 3A-D, another minimally invasive valve cutting or removal apparatus is shown accordance with the present invention and generally designated with reference numeral 200. Valve removal apparatus 200 generally includes a housing 202 and plunger 220 slidably mounted therein.

Housing 202 includes a first tubular portion or member 204, which has an annular cutting edge or element 206 at the distal end thereof, and a second portion or member 208 coupled thereto or integrally formed with first portion or member 204. In the illustrative embodiment, first and second portions or members 204 and 206 are rotatably coupled to one another through an annular tongue 210 and groove 212 arrangement as shown in FIGS. 3B-D. However, other coupling arrangements can be used and members 204 and 206 can be fixedly secured to one another or integrally formed as noted above. Second member or portion 208 includes a chamber forming housing 214 that houses and supports spring 216 and includes vertically aligned holes 218 through which plunger 220 is slidably mounted.

Referring to FIG. 3B, plunger 220 includes an elongated member or rod 222 having an enlarged disc shaped portion 224 for interfacing with spring 216, a handle or knob 226 and a cutting and leaflet holding member 228 that cooperates with cutting edge 206. In the illustrative embodiment, cutting and leaflet holding member 228 includes conical section 230 and cylindrical section 232, which forms annular cutting block or surface 234 (see e.g., FIG. 3C). Annular surface or element 234 cooperates with annular cutting edge or element 206 to cut the valve leaflets (see e.g., FIG. 3D). Cutting elements 206 and 234 can be of any suitable material such as stainless steel. As is the case with apparatus 100, the other noncutting elements of apparatus 200 can be plastic, stainless steel or any other suitable material.

In use, the distal portion of leaflet removal apparatus 200, which is adapted for passage through an aortotomy, is passed through such an aortotomy and positioned above the aortic valve leaflets a shown in FIG. 3B. Referring to FIG. 3C, the plunger is pressed, moved or translated to position plunger cutting block 234 below the aortic leaflets. Then, compression spring 216 is allowed to return toward its relaxed state to drive the plunger proximally and squeeze the leaflets between surface 234 and cutting edge 206. In this position, housing portion 204 is rotated, as indicated with the arrow in FIG. 3D, to cut the leaflets. The cut leaflets fall into conical section or holder 230, which holds the cut leaflets as apparatus 200 is removed from the aortotomy.

According to another aspect of the invention, valve prosthesis delivery apparatus is provided to rapidly deliver the valve prosthesis to the surgical site and to secure the prosthesis at the desired location.

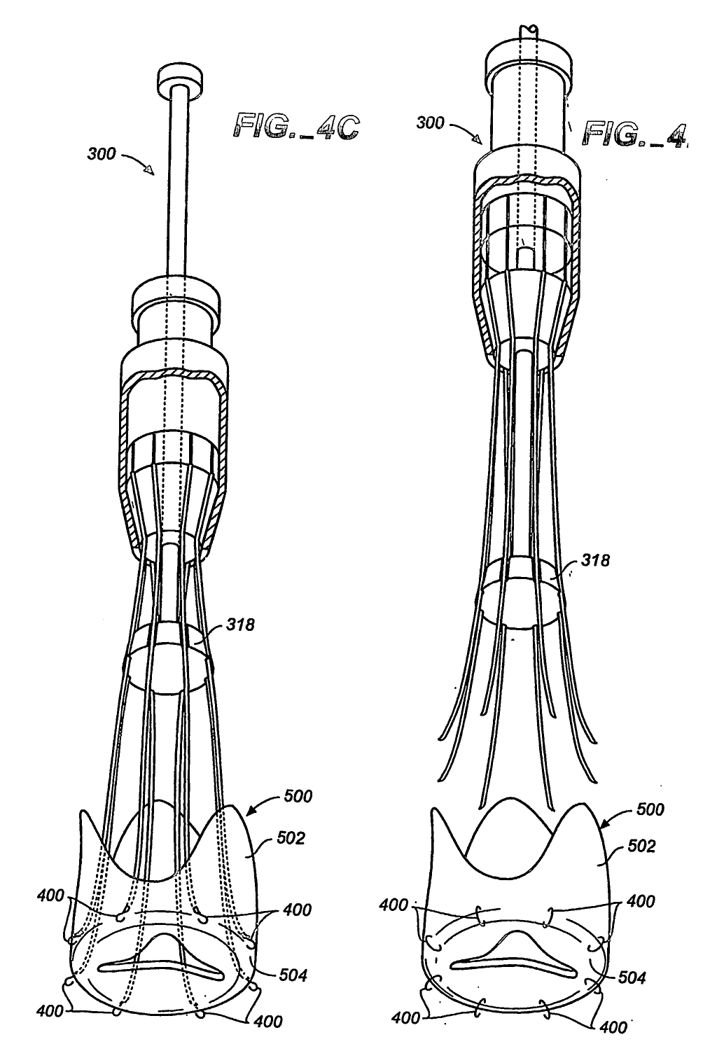

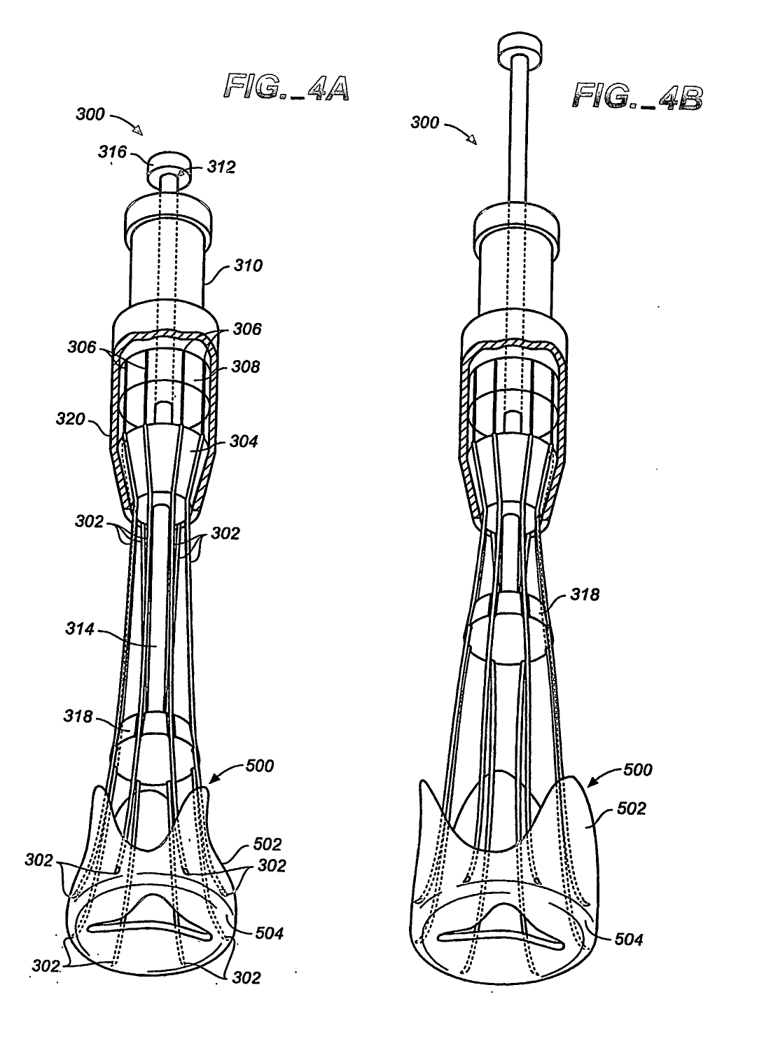

Referring to FIGS. 4A-C, an exemplary embodiment of a valve prosthesis delivery mechanism or apparatus, which is generally designated with reference numeral 300, is shown. Valve prosthesis delivery apparatus 300 generally includes a support for supporting the prosthesis and a plurality of fasteners ejectably mounted in the support.

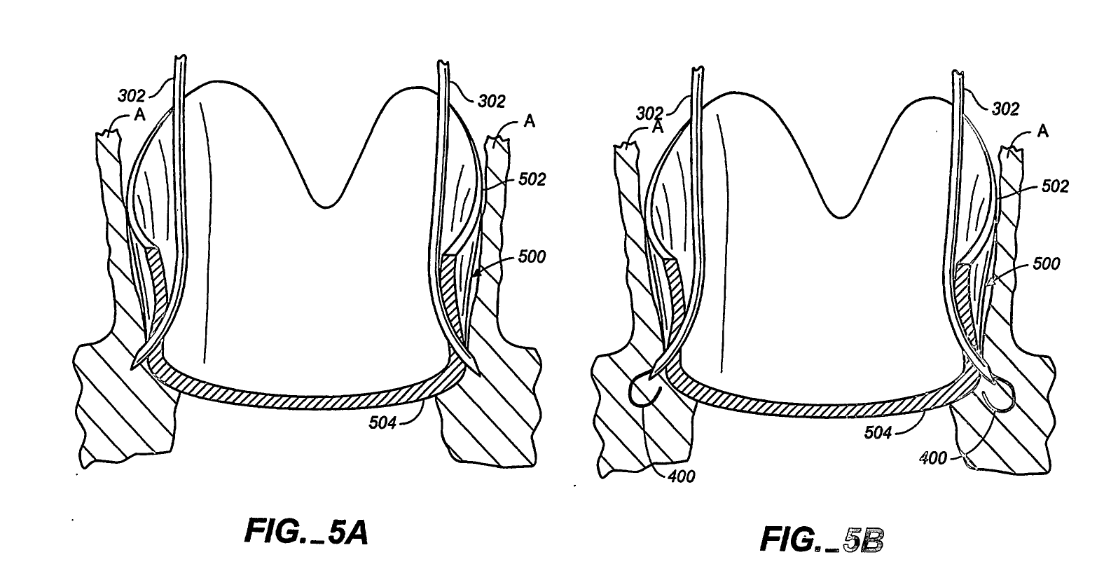

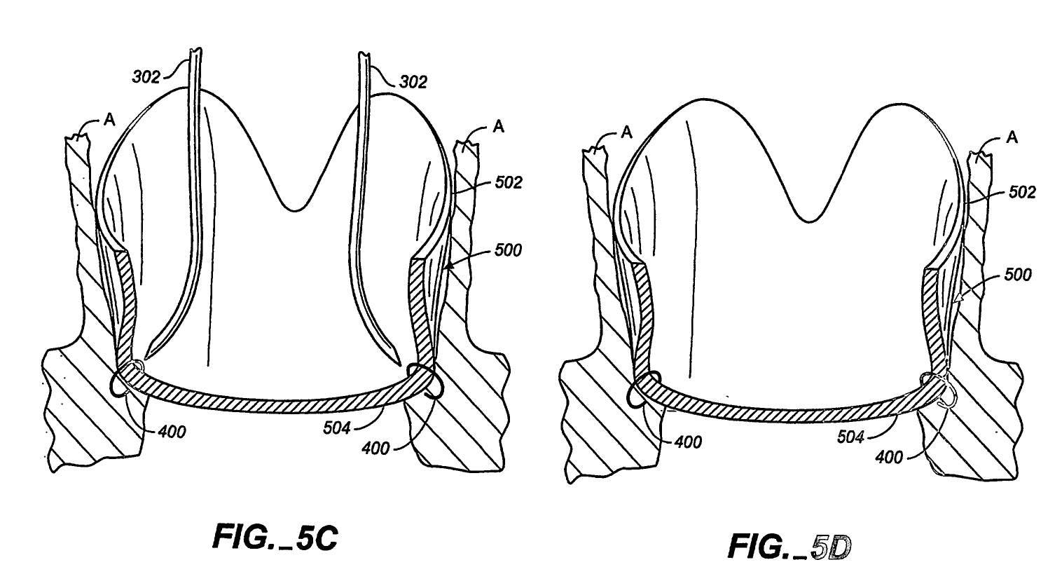

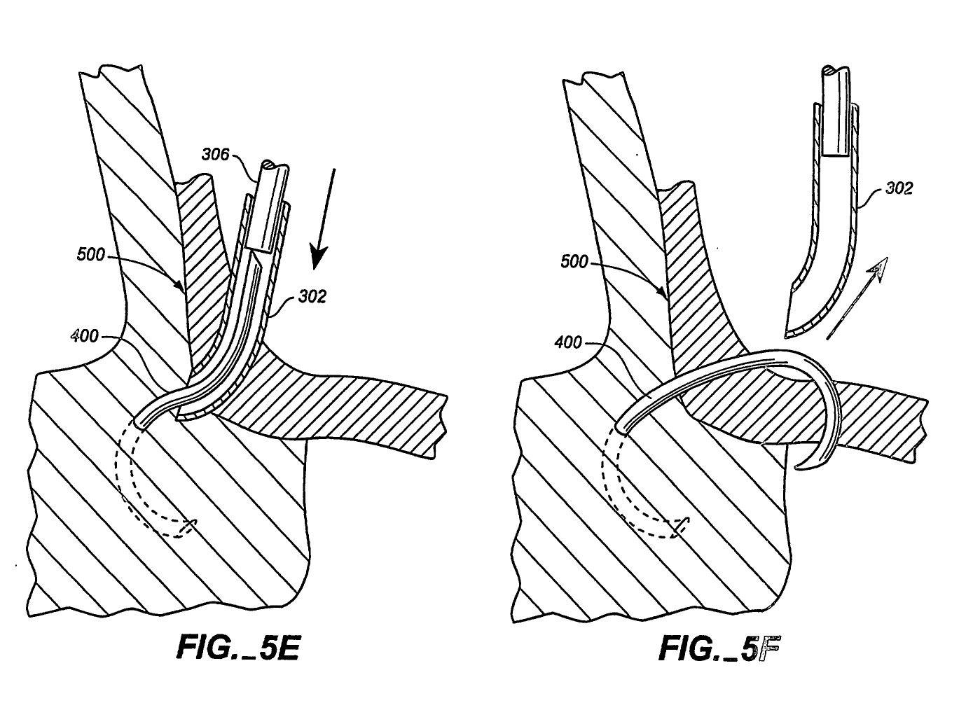

Referring to FIG. 4A, valve prosthesis mechanism 300 includes a prosthesis support comprising a plurality of tubes 302, each having a free distal end and a proximal portion fixedly secured to member 304, which in the illustrative embodiment, is frustoconical. A wire or pusher 306 is slidably mounted in each support tube 302 and includes a proximal portion that extends therefrom and is fixedly secured to plug 308, which can have the disc shape shown in the drawings. Grooves can be formed in member 304 and plug 308 for receiving support tubes 302 and wires 306, which can be formed form metal such as stainless steel, which has desirable stiffness. However, other suitable materials including nitinol can be used. The other components of apparatus 300 can be plastic, stainless steel or any other suitable material. Tubes 302 and wires 306 can be secured in the grooves by sizing the grooves to be slightly smaller than the tubes and/or wires and compressing the tubes and/or wires in the grooves and/or by gluing. Accordingly, the grooves can be made slightly larger than the tubes and wires and glue applied to hold the tubes and wires therein. Plug 308 can be secured to cylindrical member 310 or integrally formed therein and form a portion thereof. Accordingly, when cylindrical member 310 is moved distally, wires 306 move distally to eject fastener clips 400 from support tubes 302 as shown, for example, in FIGS. 4C, 5E and 5F.

Valve prosthesis delivery apparatus 300 also can include apparatus or a mechanism for expanding support tubes 302 radially outward. In the illustrative embodiment, apparatus 300 includes a plunger 312, which includes elongated member 314. Elongated member 314 has a knob 316 at its proximal end and a slide member 318 at its distal end. Slide member 318 has a plurality of grooves formed therein in which support tubes 302 are slidably mounted. Slide member 318 is sized and/or configured so that when plunger 312 is moved proximally with slide member 318, slide member 318 urges support tubes 302 radially outward.

Plug 308 can be slidably mounted in a tubular housing 320, which can be secured to frustoconical member 304 as shown in the drawings. Housing 320 also is configured to slidably receive cylinder 310.

In use, valve prosthesis such as valve prosthesis 500 is secured to valve prosthesis delivery apparatus 300. Valve prosthesis 500 is schematically shown as a conventional stentless tissue valve, which can be harvested from a suitable animal heart such as a porcine heart and prepared according to known methods. Valve prosthesis 500 includes a root portion 502 and a valve leaflet portion 504, which leaflet portion is shown in the drawings in an open position. In a closed configuration, the valve leaflet edges coapt to seal the valve and prevent regurgitation.

When securing valve prosthesis 500 to delivery apparatus 300, sliding member 318 is moved distally to allow the support tubes to return to their radially inward biased position as shown in FIG. 4A. Valve prosthesis 500 is then mounted on apparatus 300 so that the sharp pointed distal end of each support tube 302 extends through the lower wall portion or lower portion of root portion 502 of tissue valve prosthesis 500.

Referring to FIGS. 4A-D, use of apparatus 300 is schematically shown. FIG. 4A illustrates how sliding member 318 can be advanced to allow the support arms to move radially inward to a collapsed state as a result of the biasing effect of frustoconically shaped member 304. This position is used to introduce the apparatus through an aortotomy to the surgical site. FIG. 4B shows sliding member 318 retracted to place the arms in a radially expanded state. FIG. 4C shows cylinder 310 moved distally to eject the fastener clips 400, which are self-closing clips and fasten the valve prosthesis to the heart (not shown). FIG. 4D illustrates removal of the delivery apparatus after the clips have been released.

Self-closing clips 400 can comprise wire made from shape memory alloy or elastic material or wire so that they tend to return to their memory shape after being released from the clip delivery apparatus. As is well known in the art, shape memory material has thermal or stress relieved properties that enable it to return to a memory shape. For example, when stress is applied to shape memory alloy material causing at least a portion of the material to be in its martensitic form, it will retain its new shape until the stress is relieved as described in U.S. Pat. No. 6,514,265 to Ho, et al., entitled “Tissue Connector Apparatus with Cable Release” and U.S. Pat. No. 6,641,593, entitled “Tissue Connector Apparatus and Methods,” the disclosures of which are hereby incorporated herein by reference. Then, it returns to its original, memory shape. Accordingly, at least a portion of the shape memory alloy of each clip 400 is converted from its austenitic phase to its martensitic phase when the wire is in its deformed, open configuration inside the curved distal end portion of a respective tube 302 (see e.g., FIG. 5E). When the stress is removed and a respective clip 400 unrestrained, the material undergoes a martensitic to austenitic conversion and the clip springs back to its undeformed configuration ( FIG. 11). One suitable shape memory material for the clip 400 is a nickel titanium (nitinol) based alloy, which exhibits such pseudoelastic (superelastic) behavior.

The nitinol may include additional elements which affect the yield strength of the material or the temperature at which particular pseudoelastic or shape transformation characteristics occur. The transformation temperature may be defined as the temperature at which a shape memory alloy finishes transforming from martensite to austenite upon heating (i.e., A_{f} temperature). The shape memory alloy preferably exhibits pseudoelastic (superelastic) behavior when deformed at a temperature slightly above its transformation temperature. As the stress is removed, the material undergoes a martensitic to austenitic conversion and springs back to its original undeformed configuration. In order for the pseudoelastic wire to retain sufficient compression force in its undeformed configuration, the wire should not be stressed past its yield point in it deformed configuration to allow complete recovery of the wire to its undeformed configuration. The shape memory alloy is preferably selected with a transformation temperature suitable for use with a stopped heart condition where cold cardioplegia has been injected for temporary paralysis of the heart tissue (e.g., temperatures as low as 9-10 degrees Celsius).

The clip can be made by wrapping a nitinol wire having a diameter in the range of about 0.003 to 0.015 inch, and preferably 0.010 inch, and wrapping it around a mandrel having a diameter in the range of about 0.020 to 0.150 inch, and preferably 0.080 inch. The heat treatment of the nitinol wire to permanently set its shape as shown in FIG. 11 can be achieved by heat-treating the wire and mandrel in either a convection oven or bath at a temperature range of 400 to 650° C., preferably 520° C., for a duration of 1 to 45 minutes, and preferably 15 minutes.

The following example is set forth with reference to FIGS. 5A-5E, 6, and 7 to further illustrate operation of valve prosthesis delivery apparatus 300 in replacing a malfunctioning aortic valve. It should be understood, however, that this example is not intended to limit its scope of the invention.

A patient is placed on cardio-pulmonary bypass and prepared for open chest/open heart surgery, which typically requires a sternotomy. The surgeon removes the aortic leaflets using valve removal apparatus 100 or 200 as described above. Once the valve has been excised and removed with the valve removal apparatus, the surgeon then places a conventional aortic sizer through the aortotomy to determine the size of the aortic valve replacement (e.g., valve prosthesis 500) as is known in the art.

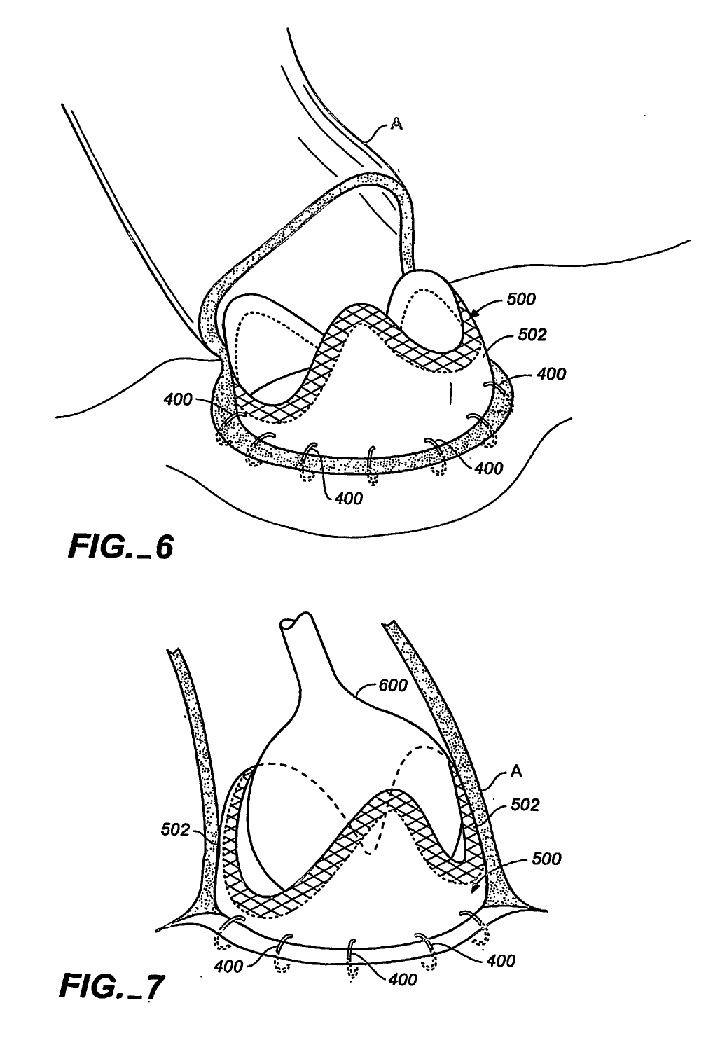

While in the generally collapsed state shown in FIG. 4A, valve prosthesis apparatus 300 with prosthetic valve 500 secured thereto is introduced through the aortotomy. The valve aligned with its natural location just below the two coronary arteries as is known in valve surgery. The sliding member 318 is retracted to have the piercing ends of support tubes 302 penetrate into the aortic root tissue as shown in FIG. 5A. The piercing ends further penetrate through the aortic root of prosthesis 500 and penetrate into the natural aortic root surrounding aortic root 502 of valve prosthesis 500. With valve prosthesis 500 seated and the sharp distal ends of the support arms 302 penetrated through the sides of the replacement valve 500 and slightly pushed further into the adjacent wall tissue of the natural aortic root, clips 400 are ejected into the adjacent wall tissue as diagrammatically shown in FIG. 5B. In this manner, the clips can penetrate the valve annulus, which is part of the aortic root. Specifically, cylinder 310 is moved distally so that pushers or wires 306 eject all of the clips 400 simultaneously (see FIGS. 4C and 5E). This one shot clip delivery can significantly reduce the time required to implant valve prosthesis as compared to other known techniques. After the clips are fully released and have moved or tended to move toward their memory shape to secure valve prosthesis 500 in place as diagrammatically shown in FIG. 5C and more particularly in FIG. 5F, valve prosthesis delivery apparatus 300 is removed leaving the replacement valve secured at the desired site (FIG. SD). FIG. 6 illustrates how the valve prosthesis attachment would appear if the aortic root were cut and pulled back after implantation. The clips anchor the stentless aortic valve into the aortic root.

Referring to FIG. 7, a conventional aortic balloon catheter including a balloon, such as balloon 600, is used to urge the outer surface of the root of the valve prosthesis against the inner wall of the aorta. Before introducing the valve prosthesis through the aortotomy, the outer surface of the root of the valve prosthesis is coated with bio-glue. Accordingly, as the balloon is expanded, it compresses the outer wall surface of the prosthetic aortic root and the bio-glue applied to the prosthetic aortic root against the natural aortic inner wall (inner wall of the natural aortic root) and it can hold it there while the glue sets. The glue can be applied to the prosthetic aortic root after the prosthetic valve is secured to the aortic valve. Depending on the glue used, a polymerizing agent may be used to activate the glue as is known in the art. As is conventional in the art, the bio-glue can be applied to form a narrow margin along the uppermost portion of the prosthetic valve root as schematically shown in cross-hatching in FIG. 6. After the glue sets, the balloon is deflated and removed from the aortotomy and the aortotomy closed by conventional means.

Although the foregoing method has been described in connection with open chest surgery, the leaflet removal apparatus and prosthesis delivery apparatus described herein can be used with minimally invasive approaches that typically require a thoracotomy between adjacent ribs. Further, although the minimally invasive valve prosthesis replacement procedure has been described with reference to one prosthetic tissue valve, it should be understood that variations of such prosthesis or other valve prosthesis types can be used. FIG. 10 illustrates valve prosthesis in place over an aortic valve after delivery with apparatus 300. Clips 400 penetrate through ring 400 and the aortic root of aorta A.

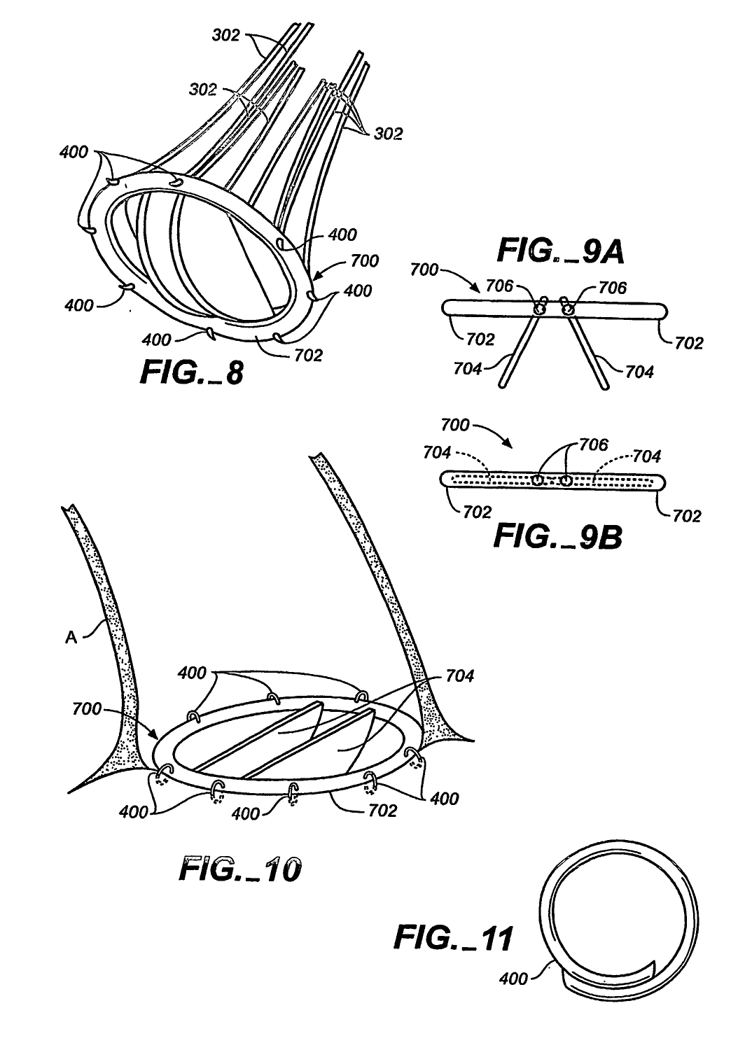

Referring to FIG. 8, Valve prosthesis delivery apparatus 300 is shown in combination with a conventional mechanical heart valve prosthesis generally designated with reference numeral 700. Mechanical heart valve prosthesis 700 comprises an annular ring or housing 702, which can be metal or carbon material, to which two valve leaflets 704 are pivotally mounted. Each leaflet is pivotally mounted to ring 702 with two pivots 706 (two of the four pivots being hidden from view in FIG. 9A). A portion of each leaflet extends beyond its respective pivot as shown in FIG. 9A so that the leaflets can fully close the valve opening that ring 702 forms. Atlhough a particular mechanical heart valve prosthesis is shown, it should be understood that any suitable mechanical heart valve prosthesis (or other valve prosthesis) can be used without departing from the scope of the invention. For example, a mechanical valve having a ball can be used. Such ball valves also are known in the art.

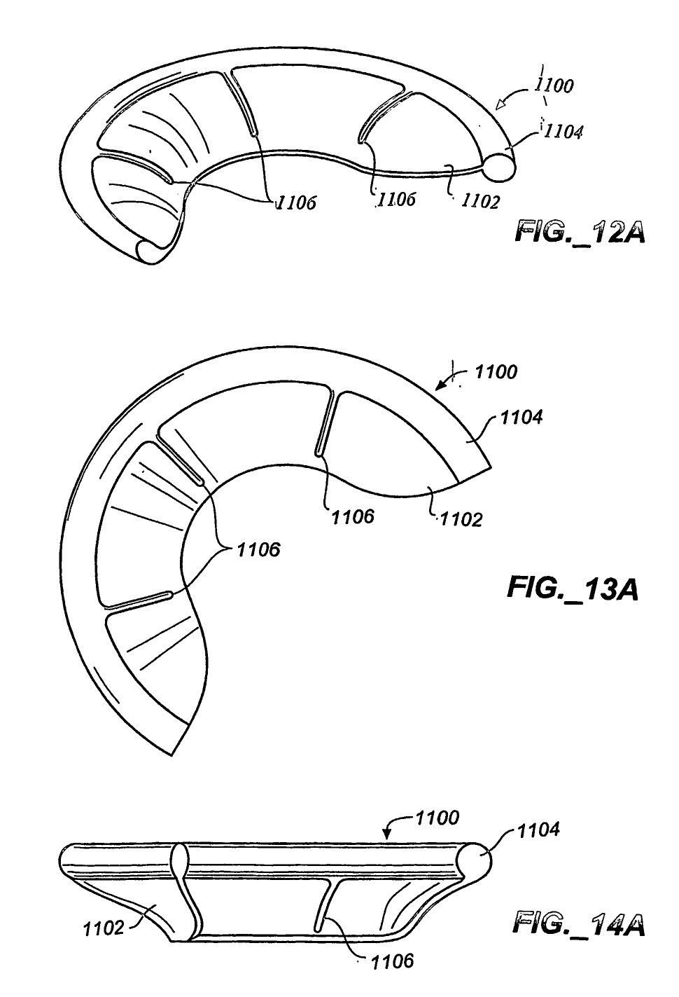

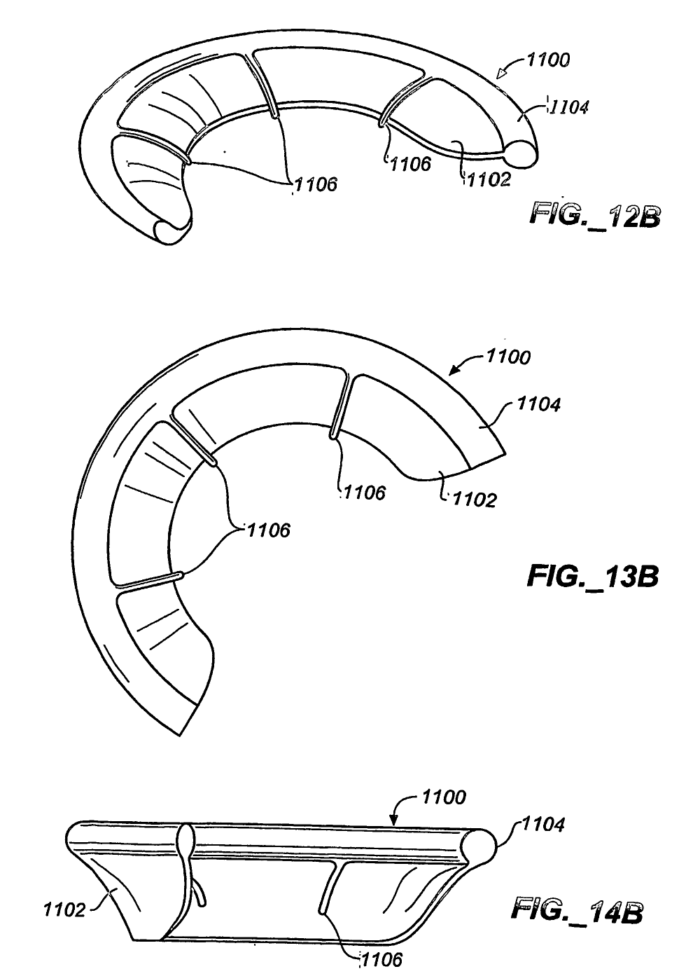

Referring to FIGS. 12A and B, 13A and B and 14A and B, plan and side view of one embodiment of a valve prosthesis, generally designated with reference numeral 1100 and including a replacement valve leaflet is shown in accordance with the principles of the present invention. The replacement valve leaflet is shown in a closed configuration in FIGS. 12A, 13A, and 14A, and in an open configuration in FIGS. 12B, 13B, and 14B.

Referring to 12A, 13A and 14A, exemplary valve prosthesis 1100 includes a skirt or prosthetic leaflet 1102, which is configured to replace or extend over and cover a leaflet in the valve under repair (e.g., the mitral valve posterior leaflet). Skirt or valve leaflet 1102 can, for example, be made from ePTFE or prosthetic tissue. One prosthetic tissue that can be used is pig leaflet tissue. When repairing a mitral valve, the skirt can be configured to cover the posterior leaflet and effectively replace the posterior leaflet without removing it.

Skirt 1102 is secured to a member or mechanism for holding it in the desired location. In the illustrative embodiment, skirt 1102 is secured to curved member 1104, which can be in the form of an open or partial annuloplasty ring. Skirt 1102 can be secured to ring 1104 by gluing, using conventional medical gluing materials, or sewing or it can be wrapped around ring 1104 and glued or fused to itself. Although not shown, it should be understood that the curved member also can be in the form of a fill, continuous or closed annuloplasty ring.

Member 1104 can be made from any suitable material(s) such as from one or more biocompatible polymers including but not limited to silicone. It also can be covered with Dacron® material such as synthetic polyester textile fiber material or fibrous mesh to assist with tissue ingrowth after implantation. Further, curved member 1104 can be rigid or flexible. Rigid or nonpliable rings, whether full or partial, can improve the ability to reshape the mitral valve annulus. Flexible rings, whether fill or partial, can more readily conform to the mitral valve annulus and accommodate valve movement. In the case where curved member 1104 is to be rigid or nonpliable, suitable plastics can be used. Alternatively, it can be reinforced with a stainless steel or titanium insert(s), which can be in the form of threads or wires extending generally parallel to the longitudinal axis of the curved member, e.g., curved member 1104.

Curved member 1104 also can be provided with a plurality of struts 1106 that extend radially therefrom in an inward direction and provide reinforcement or support for skirt 1102. More specifically, the struts can be curved radially inward and downward to conform to the surface or curvature of replacement leaflet 1102 when replacement leaflet 1102 is in its desired closed position during diastole. The struts, which can be made from the same material as member 1104, can be attached to curved member 1104 or integrally formed therewith, but are not attached to skirt 1102 so that the skirt can move away form the struts during diastole and toward or to the struts during systole. Since the replacement valve leaflet does not have chordae tendineae, the struts are provided to prevent the replacement valve leaflet from folding backward during the systolic cycle. The struts, however, do not extend completely to the inner perimeter of skirt 1102 (see e.g., FIG. 12A). The inner circumferential margin of the skirt that extends inwardly beyond the struts facilitates contact or apposition between the skirt and the opposed leaflet to effect a seal therebetween during systole. Otherwise, one or more of the struts may contact the opposed leaflet and form a gap and cause regurgitation. The inner circumferential margin can range from about 1 to 3 mm.

The prosthesis can be secured to the valve by suturing or the use of clips or other fasteners. It can simply be placed on the desired location of the valve and the fasteners placed to secure the prosthesis to the valve. Examples of suitable clips are described in, but not limited to, U.S. Pat. No. 5,972,024 to Northrup, et al. and entitled “Suture-Staple Apparatus and Method,” U.S. Pat. No. 6,514,265 to Ho, et al. and entitled “Tissue Connector Apparatus with Cable Release,” and U.S. Pat. No. 6,613,059 to Schaller, et al. and entitled “Tissue Connector Apparatus and Methods,” the disclosures of which are hereby incorporated herein by reference. Alternatively, the prosthesis can be more rapidly secured to the valve using clip delivery apparatus and/or valve prosthesis delivery apparatus constructed according to further aspects of the invention.

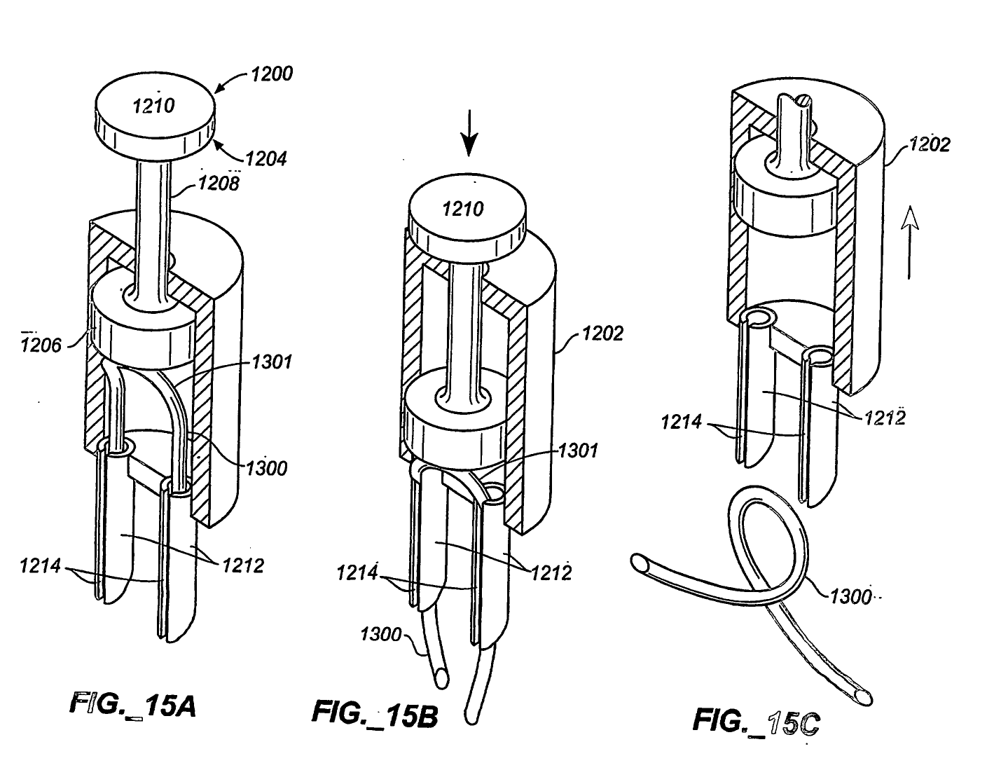

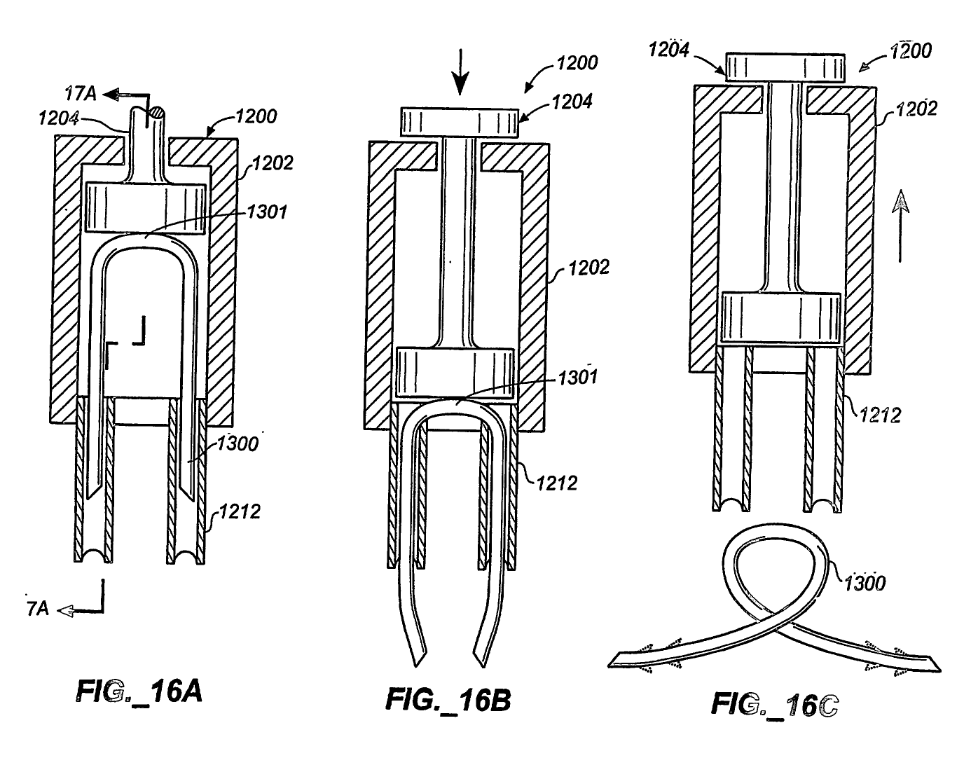

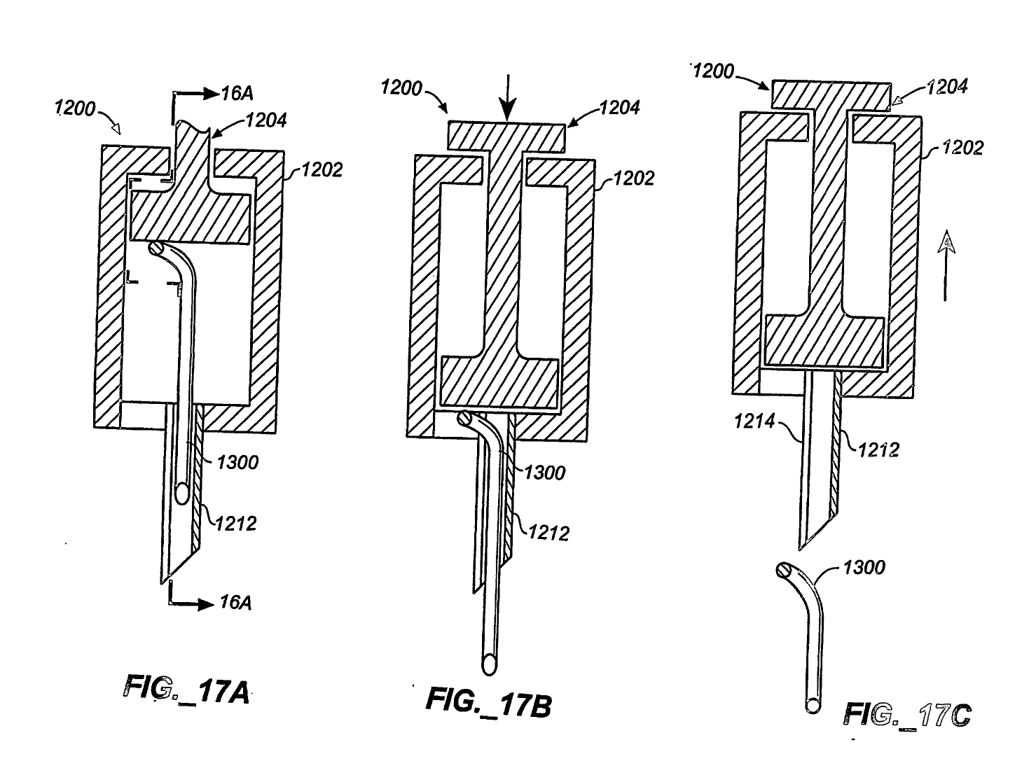

FIGS. 15A-C are partial sectional views of one exemplary embodiment of clip delivery apparatus, which is generally designated with reference numeral 200, for ejecting fasteners through the prosthesis and securing the prosthesis a patient's valve. Apparatus or mechanism 1200 includes a cylindrical housing 1202 and an ejector or plunger 1204 slidably mounted therein. Plunger 1204 includes a piston head 1206 and a piston rod 1208 extending therefrom and terminating in an actuator member or anvil 1210. Clip delivery apparatus 1200 further includes fastener guide tubes 1212, which can be hypotubes and which can have longitudinal slots 1214 extending therethrough. Each guide tube can be integrally formed with housing 1202 or they may be separately formed and secured to the housing by gluing or welding. Referring to FIGS. 15A-C, 16A-C, and 17A-C, as the anvil is pressed and the piston nears or contacts the guide tubes, the self-closing clip shown in the drawings is ejected and if unrestrained, returns to its relaxed state as shown in FIGS. 15C, 16C, and 17C. Specifically, when each clip is restrained in a respective guide tube 1212, the upper end of each clip 1300, is angulated forward and is outside the guide tubes as shown, for example, in FIGS. 15A-4B and 16A-B. This angulated portion of the clip, which also joins the illustrated generally straight clip portions, is designated with reference numeral 1301. As piston head 1206 is pushed distally, it pushes angulated portion 1301, which then pulls the portions adjacent thereto therewith and out of slots 1214 (see e.g., FIG. 16B). Once those portions of the clip begin to come out through slots 1214, the remainder of the clip follows because the clip is spring loaded in the tubes and wants to return to its memory shape or free state.

One fastener that can be used with clip delivery apparatus is a self-closing clip. One such clip is shown in its open, deformed configuration in FIG. 1SA and in a relaxed, free state or closed configuration in FIG. 15C. The illustrative clip of FIG. 15C can be described as having a closed loop configuration. The clip is generally designated with reference numeral 1300. Clip 1300 has pointed or sharpened ends for piercing through curved member 1104 and the valve annulus as will be described in more detail below. Further, clip 1300 can have barbs as shown in dashed line in FIG. 16C to enhance securement of the prosthesis to the valve annulus.

The clip can comprise wire made from shape memory alloy or elastic material so that it tends to return to its memory shape after being released from the clip delivery apparatus. As is well known in the art, shape memory material has thermal or stress relieved properties that enable it to return to a memory shape. For example, when stress is applied to shape memory alloy material causing at least a portion of the material to be in its martensitic form, it will retain its new shape until the stress is relieved as described in U.S. Pat. No. 6,514,265 to Ho, et al. and entitled “Tissue Connector Apparatus with Cable Release” and U.S. Pat. No. 6,641,593 to Schaller, et al. and entitled “Tissue Connector Apparatus and Methods,” the disclosures of which are hereby incorporated herein by reference. Then, it returns to its original, memory shape. Accordingly, at least a portion of the shape memory alloy of clip 1300 is converted from its austenitic phase to its martensitic phase when the wire is in its deformed, open configuration (see e.g., FIG. 15A). As the stress is removed, the material undergoes a martensitic to austenitic conversion and springs back to its undeformed configuration (see e.g., FIG. 15C). One suitable shape memory material for the clip 1300 is a nickel titanium (nitinol) based alloy, which exhibits such pseudoelastic (superelastic) behavior.

The nitinol may include additional elements which affect the yield strength of the material or the temperature at which particular pseudoelastic or shape transformation characteristics occur. The transformation temperature may be defined as the temperature at which a shape memory alloy finishes transforming from martensite to austenite upon heating (i.e., A_{f} temperature). The shape memory alloy preferably exhibits pseudoelastic (superelastic) behavior when deformed at a temperature slightly above its transformation temperature. As the stress is removed, the material undergoes a martensitic to austenitic conversion and springs back to its original undeformed configuration. In order for the pseudoelastic wire to retain sufficient compression force in its undeformed configuration, the wire should not be stressed past its yield point in it deformed configuration to allow complete recovery of the wire to its undeformed configuration. The shape memory alloy is preferably selected with a transformation temperature suitable for use with a stopped heart condition where cold cardioplegia has been injected for temporary paralysis of the heart tissue (e.g., temperatures as low as 9-10 degrees Celsius).

The clip can be made by wrapping a nitinol wire having a diameter in the range of about 0.002 to 0.015 inch, and preferably 0.011 inch, and wrapping it around a mandrel having a diameter in the range of about 0.050 to 0.150 inch, and preferably 0.100 inch. The heat treatment of the nitinol wire to permanently set its shape as shown in FIG. 15C can be achieved by heat-treating the wire and mandrel in either a convection oven or bath at a temperature range of about 400 to 600° C., preferably 450° C., for a duration of about 1 to 45 minutes, preferably about 15 minutes.

According to another aspect of the invention, valve prosthesis delivery apparatus is provided to rapidly deliver the valve prosthesis to the surgical site and to secure the prosthesis at the desired location.

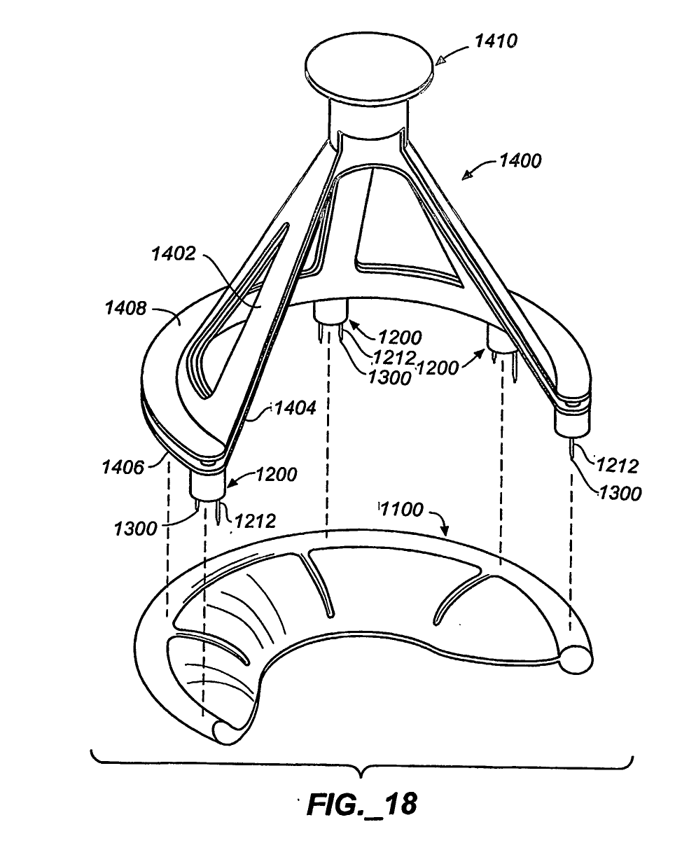

Referring to FIG. 18, an exemplary embodiment of a valve prosthesis delivery mechanism, which is generally designated with reference numeral 1400, is shown. Valve prosthesis delivery apparatus 1400 includes a first member 1402 slidably or movable coupled to a second member 1404. Members 1402 and 1404 are shown as being in a frustoconical shape with cut outs to enhance visibility of the surgical site and lighten the apparatus. Members 1402 and 1404 also are configured so that member 1404 fits within member 1402. In the example provided in FIG. 18, member 1404 is nested in member 1402. Alternatively speaking, member 1402 is stacked on member 1404.

Second member 1404 includes a clip delivery support(s) for supporting a plurality of clip delivery devices 1200. In the illustrative embodiment, a clip delivery support is shown in the form of a partial flat ring 1406. Ring 1406 has a plurality of holes formed therein in which piston rods 1208 of clip delivery apparatus 1200 or devices are disposed. First member 1402 includes a head(s) or anvil(s) adapted to push clip ejectors 1204 in a distal direction to eject clips 300. In the illustrative embodiment, a first member head or anvil is shown in the form of a partial flat ring 1408. First member 1402 also includes a plunger knob or grip 1410 to push member 1402 downwardly when the prosthesis delivery apparatus is positioned over the surgical site as will be discussed in more detail below. Grip 1410 can be in the form of a cylinder with a cap at one end (a closed end cylinder) extending from the frustoconical body portion of first member 1402 as shown in FIG. 7.

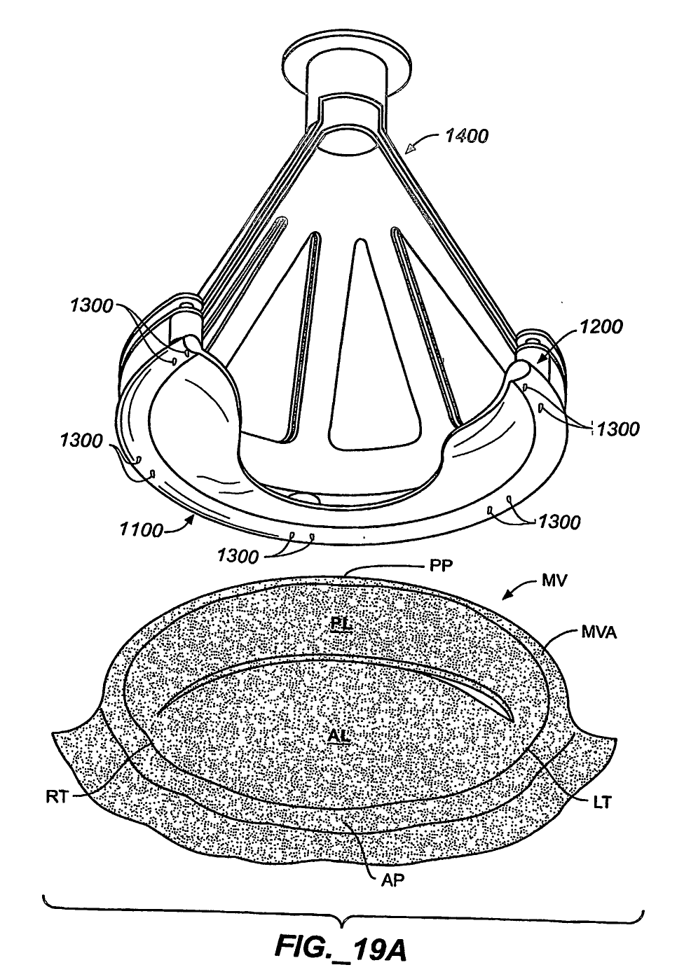

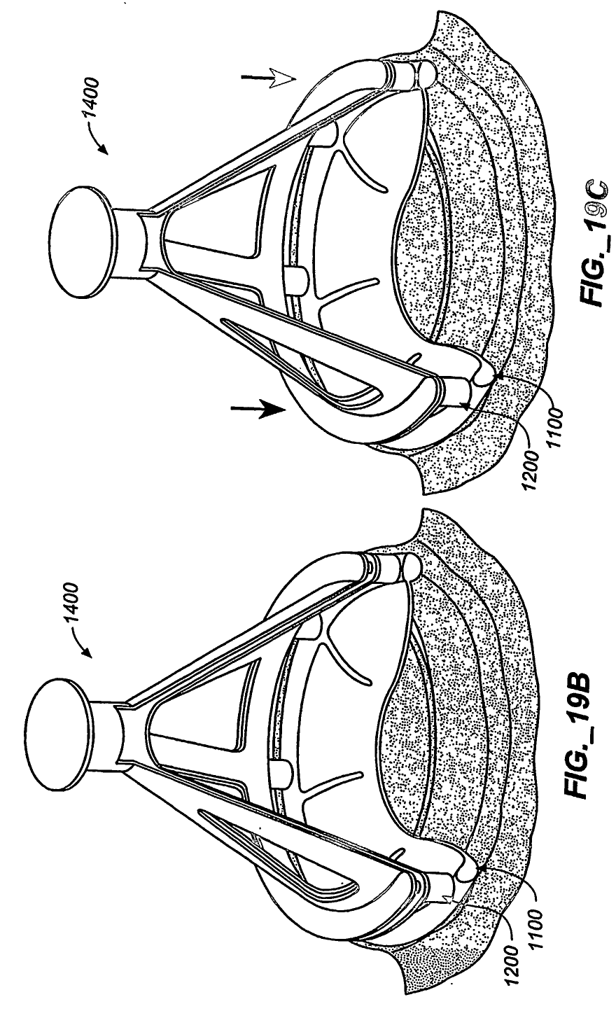

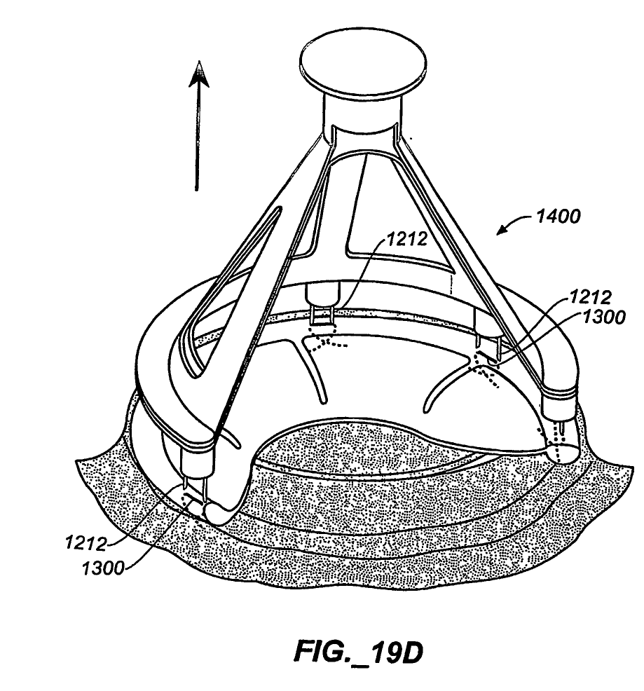

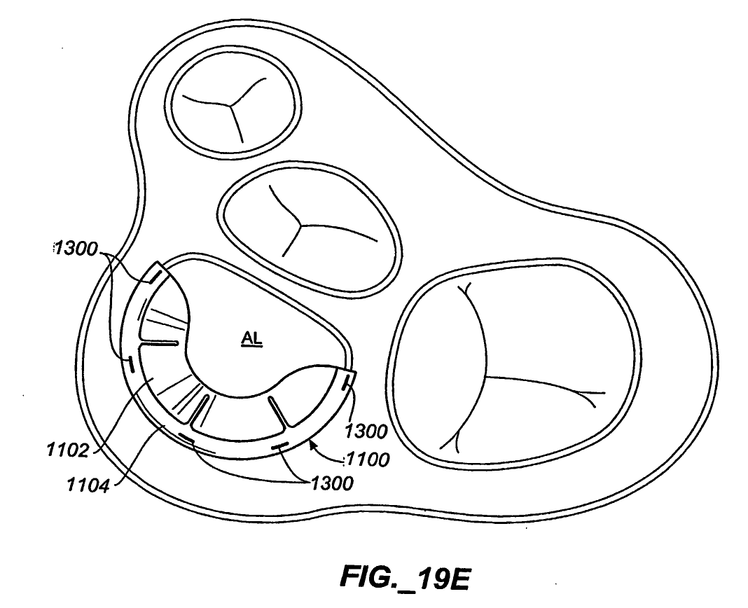

When clips 1300 are positioned in clip delivery apparatus 1200 in an open, deformed configuration as shown, for example, in FIGS. 15A and 16A, the clips maintain the ejectors in a proximal position or loaded position with rings 1406 and 1408 spaced from one another as shown, for example, in FIG. 18. Guide tubes 1212 restrain the clips in the illustrated open configuration and the interaction of the restraining force of guide tubes 1212 and the tendency of the clips to return to their relaxed state maintains the clip delivery apparatus in the position shown in FIGS. 15A and 15B and valve prosthesis delivery apparatus 1400 in the position shown, for example, in FIGS. 18, 19A, and 19B until additional force is placed on ejector heads or anvils 1210 (FIG. 19C). The materials used for valve prosthesis delivery apparatus 1400 can include a combination of plastic and metal materials suitable for medical use. For example, clip delivery apparatus 1200, ring 1406 and anvil 1408 can be medical grade stainless steel and the remaining components of delivery apparatus 1400 can be plastic such as polyurethane or polycarbonate material. Alternatively, apparatus 1200 can be stainless steel and the remaining components of apparatus 1400 can be made of the foregoing plastic material.

Although particular configurations have been shown regarding first and second members 1402 and 1404 and the clip delivery support and anvil members, other configurations can be used without departing from the scope of the invention. For example, the clip delivery support and anvil members can be full rings.

The following example is set forth to illustrate operation of the invention, and is not intended to limit its scope. Referring to FIGS. 19A-19E, an exemplary method of using prosthesis 100 to treat mitral valve insufficiency is shown in accordance with the present invention.

As noted above, a competent mitral valve (MV) allows one-way flow of oxygenated blood that has entered the left atrium from the lungs to enter the left ventricle. The left ventricle then pumps the oxygenated blood to the rest of the body.

Referring to FIG. 19A, the mitral valve (MV) comprises a pair of leaflets, the anterior leaflet (AL) and the posterior leaflet (PL) of which the latter is larger. The base of each leaflet is attached to the mitral valve annulus (MVA). The mitral valve annulus includes a posterior portion (PP) and an anterior portion (AP) also known as the inter-trigone section, which is a generally straight substantially rigid section. The posterior portion of the annulus is a flexible, curved section that encompasses a larger portion of the annulus circumference than the anterior portion. The right and left fibrous trigones (generally indicated with reference characters RT and LT) mark the end of the generally straight section (inter-trigone section) and define the intersection points between the posterior and anterior portions (PP, AP).

The leaflets open and close in response to pressure differences on either side thereof. However, when the leaflets do not fully close, regurgitation and valve insufficiency can result. One method to treat the insufficiency using the implant or prosthetic apparatus of FIG. 12A will be described with reference to FIGS. 19B-19E.

A patient is placed on cardio-pulmonary bypass and prepared for open chest/open heart surgery, which typically requires a sternotomy. The surgeon opens the left atrium of the heart and measures the size and shape of the mitral valve annulus. A valve prosthesis 1100 is selected based on the measured size and shape of the annulus so that ring or partial ring 1104 will conform to the size and shape of the annulus. Accordingly, the size and shape of curved member 1104 is selected to match the size and shape of that portion or all of the annulus upon which it is to be seated. The diameter of curved member 1104 can range form about 18 mm to about 45 mm, and more typically will range from abut 24 mm to about 36 mm. In the case where a partial ring such as illustrative member 1104 is used, the curved member is selected so that it is sized and configured for attachment to the posterior portion of the mitral valve annulus of the patient's heart. The curved member 1104 can then minimize or prevent further dilation of the annulus, while the replacement leaflet 1102 corrects the mitral regurgitation. In this manner, valve prosthesis 100 can simplify valve repair procedures.

The selected valve prosthesis is then aligned with the exposed ends of clips 1300 of valve prosthesis delivery apparatus or mechanism 1400 as shown in FIG. 18 and curved or leaflet support member 1104 is pressed against the clips, while applying downward pressure to plunger 1410 so that the piercing ends of the clips pass through leaflet support member 1104 as shown in FIG. 19A. Alternatively, the clips may remain retracted until tubes 1212 contact support member 1104 after which time they are partially ejected to partially extend from the opposite side of leaflet support member as shown in FIG. 19A. With the prosthesis secured to the prosthesis delivery apparatus, the prosthesis delivery apparatus is seated on the valve annulus as shown in FIG. 19B. Plunger or knob 1410 is then pressed downwardly to move first member 1402 and ring 1406 downardly as shown in FIG. 19C to effectuate simultaneous ejection of all of the clips from the clip delivery apparatus with a single stroke or actuation step. After the clips have been ejected into the mitral valve annulus, they move toward their closed configurations to secure the valve prosthesis to the mitral valve as shown in FIG. 19D after which the prosthesis delivery apparatus is removed. The implant and delivery apparatus can provide a quick and effective way to treat mitral valve regurgitation. The implant can be attached to the posterior portion of the mitral valve annulus such that the implant skirt or prosthetic leaflet coapts with the opposed natural leaflet and skirt support member 1104 constructed to prevent further dilation of the annulus.

The implanted prosthesis shown in FIG. 19E, illustrates a top view of the valve prosthesis in place over the mitral valve with the anterior leaflet in view and in a closed position with the prosthetic leaflet or skirt covering the natural posterior leaflet. Although the natural posterior leaflet chordae tendineae remains in place so that it can still function, leaflet coaption now occurs between the natural anterior leaflet AL and the replacement posterior leaflet 1102. If the natural posterior leaflet chordae tendineae were removed, the ventricle could sag or expand further over time, which would make it less efficient.

As noted above, the annuloplasty ring or member 1102 can be constructed to strengthen the annulus and prevent any further distension of the annulus when secured thereto. Member 1102 also can be used to shorten the annulus to treat eschemic mitral regurgitation as is done with annuloplasty rings. In this case, valve prosthesis member 1100 would not be delivered with valve prosthesis apparatus 400. Rather, the portion of member 1100 that is to be secured to the annulus would be delivered or secured to the annulus with sutures in a manner known in the art to shorten the annulus.

Variations and modifications of the devices and methods disclosed herein will be readily apparent to persons skilled in the art. As such, it should be understood that the foregoing detailed description and the accompanying illustrations, are made for purposes of clarity and understanding, and are not intended to limit the scope of the invention, which is defined by the claims appended hereto.

1. (canceled)

2. Heart valve leaflet removal apparatus comprising a pair of cooperating cutting elements adapted for cutting and removing leaflets from the aortic valve in a patient's heart, one of said cutting elements being rotatably coupled to the other of said pair of cutting elements; a holder coupled to one of said cutting elements and adapted to receive the cut leaflets; and said cutting elements and holder being configured for delivery to the aortic valve leaflets through an aortotomy formed in the patient's aorta.

3-42. (canceled)
